(19) United States
(12) Patent Application Publication
(10) Pub. No.: US 2007/0118002 A1
(43) Pub. Date: May 24, 2007

(54) Conversion of alkanes having at most 5 carbonatoms to alcohols and/or aldehydes using a platinum complex catalyst

(76) Inventors: Vladimir Mikhailovich Linkov, Somerset West (ZA); Vladimir Nikolaevich Belyakov, Kiev (UA); Ludmila Alekseevna Belyakov, Kiev (UA)

Correspondence Address: LADAS & PARRY, 26 WEST 61ST STREET, NEW YORK, NY, 10023, US

(21) Appl. No.: 10/575,692

(22) PCT Filed: Oct. 12, 2004
(86) PCT No.: PCT/IB04/03319

§ 371 Date: Jan. 24, 2007

(30) Foreign Application Priority Data
Oct. 15, 2003 (ZA) 2003-8023

(51) Int. Cl. C07C 29/48

(52) U.S. Cl. 568/469.9; 568/910.5

(57) ABSTRACT

A gas-phase process is provided for catalytic conversion of a lower alkane comprising at most 5 carbon atoms to at least one produce selected from the group consisting of alcohols, aldehydes and mixtures thereof. The process comprises bringing said lower alkane into contact with an oxidising agent in the presence of a catalyst comprising at least one platinum complex. The invention also provides such catalyst for use in the process. The catalyst comprises a platinum complex having a platinum cation core. It has two ligands selected from amino ligands, hydroxyl ligands and immidazolyl ligands whereby the complex is bonded chemically to a metal oxide support. It also has four other ligands selected from hydroxyl ligands, chloro ligands and amino ligands.

THIS INVENTION relates to catalytic conversion. More particularly, the invention relates to a process for the catalytic conversion of lower alkanes to products selected from the group consisting of alcohols and aldehydes, and to a catalyst for use in the process.

According to the invention there is provided a gas-phase process for the catalytic conversion of a lower alkane comprising at most 5 carbon atoms to at least one product selected from the group consisting of alcohols, aldehydes and mixtures thereof, the process comprising bringing a said lower alkane into contact with an oxidising agent in the presence of a catalyst comprising at least one platinum complex.

The catalyst may be a heterogeneous catalyst, each platinum complex being supported on a catalyst support. The catalyst support may comprise at least one solid metal oxide, the support having a surface to which each platinum complex is chemically bonded. Each metal oxide may be selected from the group consisting of alumina (Al₂O₃), silica (SiO₂), titania (TiO₂), zirconia (ZrO₂) and molybdena (Mo₂O₃), silicon and molybdenum for this purpose being regarded as metals. The catalyst support may comprise a plurality of said metal oxides. In particular, each platinum complex may have two ligands which are chemically bonded to the surface of the metal catalyst support. The catalyst may have, for example, the following structure: 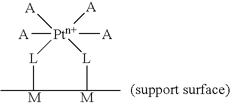
in which:

each L is a ligand selected from amino ligands, hydroxyl ligands and imidazolyl ligands;

each A is a ligand selected from hydroxyl ligands, chloro ligands and amino ligands;

each M is a metal cation of the catalyst support and is selected from the cations of aluminium, silicon, zirconium, titanium and molybdenum; and

n is an integer selected from 4, 2 and 1,

the Pt of the catalyst being a platinum cation having a valence state selected from the valence states (IV), (II) and (I), corresponding to the numerical value of n, said platinum cation forming a central core of the platinum complex, the L ligands and the A ligands surrounding the central core, and the L ligands acting chemically to bond the complex to the surface of the support, under which surface the metal cations M are located.

The catalyst, including the catalyst support, may have a platinum content of 1-6 mg/cm³, preferably 2-5 mg/cm³.

The lower alkane may be methane, the catalytic conversion being direct catalytic conversion by partial oxidation of the methane to form a product selected from the group consisting of methanol, formaldehyde and mixtures thereof. This is expected to be a particularly useful application of the process of the present invention.

The oxidising agent may be molecular oxygen (O₂), the oxidising agent being contacted in the gas phase with the lower alkane. Typically the O₂ will be a component of air or a suitable air/oxygen mixture which is contacted in the gas phase with the lower alkane. It follows that the molecular oxygen may be diluted by molecular nitrogen (N₂).

The process may be carried out at an elevated temperature. Thus the lower alkane and oxidising agent may be mixed together to form a reaction mixture, the reaction mixture being brought into contact with the catalyst at a reaction temperature which is above ambient temperature. The reaction temperature may be in the range 60-120° C., usually 90-120° C., although any temperature in the range 30-180° C. can in principle be employed, if desired.

The pressure at which the process is carried out is not critical. Thus, the reaction mixture may conveniently be brought into contact with the catalyst at ambient atmospheric pressure.

The catalyst may be supported on a solid support contained in a reactor, the reaction mixture being fed into the reactor and into contact with the catalyst at a space velocity of 2-20 cm³ reaction mixture/cm³ reactor volume/minute. In particular the catalyst may be supported on a solid support contained in a reactor, the support having a porous surface and a porous interior, and the reaction mixture being passed in the reactor over the supported catalyst and through the porous interior of the support.

The support may be in porous sheet or tube form, the support separating a reagent inlet to the reactor from a product outlet from the reactor, the reaction mixture being caused to pass through the porous interior of the support in a direction from the inlet to the outlet.

The catalyst support may in other words be porous and may be in porous sheet form, the porous sheet being flat or optionally formed into a tube so that the support is tubular, the support being fixed in the interior of the reactor, and the reaction mixture being passed through the support and over the catalyst in the reactor. As indicated above the catalyst support may comprise a plurality of said metal oxides as components thereof, being, for example, a bi-component or multi-component mixture to said metal oxides. In this regard it is to be noted, as also indicated above, that the present specification regards silica (Si) and molybdenum (Mo) to be metals. Furthermore, the catalyst support may comprise, in addition to said metal oxides, additional components such as aluminosilicates, zeolites, porous ceramic oxides, fibrous oxides, and additional metal oxides such as bi-component and multi-component metal oxides.

The invention extends to a catalyst comprising a platinum complex supported on a metal oxide support and having the structure: 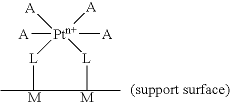
in which:

each L is a ligand selected from amino ligands, hydroxyl ligands and imidazolyl ligands;

each A is a ligand selected from hydroxyl ligands, chloro ligands and amino ligands;

each M is a metal cation of a solid catalyst support comprising at least one metal oxide selected from alumina, silica, zirconia, titania and molybdena, M correspondingly being selected from the cations of aluminium, silicon, zirconium, titanium and molybdenum; and

n is an integer selected from 4, 2 and 1,

the Pt of the catalyst being a platinum cation having a valence state selected from the valence states (IV), (II) and (I) and corresponding to the numerical value of n, said platinum cation forming a central core of the platinum complex, the L ligands and the A ligands surrounding the central core, and the L ligands acting chemically to bond the complex to the surface of the support, under which surface the metal cations M are located.

Preferably, each ligand L is selected from amino ligands and hydroxyl ligands;
each ligand A is selected from hydroxyl ligands and chloro ligands;
each metal cation M is silicon; and
n has a value of 4,
   the platinum cation of the catalyst having a valence state of (IV). As indicated above, the catalyst support may be in porous sheet form, the porosity of the sheet permitting passage of a gas-phase reaction mixture through the sheet from one side of the sheet to another. The porous sheet may be formed into one or more tubes so that the catalyst support is tubular, or it may be in flat sheet form.

The invention extends further to a product, in particular an alcohol or an aldehyde, whenever produced by the process described and defined above.

The catalyst of the present invention typically comprises polyvalent platinum as a component of a poly-ligand complex bonded to a metal oxide surface. Platinum cations can be present in three oxidation states, corresponding respectively to the valence states (IV), (II) and (I). Without being bound by theory and without the invention's being fully understood, it is believed that, during the alkane oxidation process, Pt (IV) is reduced to Pt (II) and possibly also to Pt (I). Simultaneously with alkane oxidation, the oxygen from the oxidizing agent (molecular oxygen optionally as part of air or diluted thereby) present in the reaction mixture re-oxidizes the platinum, thus returning it to the highest oxidation state (IV).

The catalyst of the present invention typically comprises the platinum complex bonded to a metal oxide surface, which acts as a catalyst support, and in this case, two ligands of the platinum complex are usually chemically bonded to surface metal atoms of the support. The structure of one of the more active supported platinum complexes, mentioned above and attached to the surface of a silica support, is as follows:

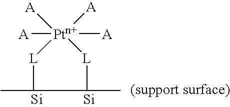

In this complex, as indicated above, L is an amino ligand or a hydroxyl ligand, A is a hydroxyl ligand or a chloro ligand, M is silicon and n is 4. According to the mechanism discussed above, platinum cations in the surface complex are present in the valence state of (IV).

As indicated above, the catalyst of the present invention typically consists of the platinum complex chemically bonded to the surface of the support. Suitable supports include the abovementioned oxides of aluminium, silicon, titanium, zirconium, molybdenum and optionally also the abovementioned other suitable metal oxides and mixtures thereof, such as aluminosilicates, zeolites, porous oxides, fibrous oxides, bi-component mixed oxides and multi-component mixed oxides.

Preferred supports are manufactured from porous metal oxides and are in the form of flat sheets or tubes. Fibrous and globular ceramic materials containing oxides of each of silicon, aluminium and zirconium can be used as starting materials for porous support preparation. The porous structure of the support can be modified by introducing additional ceramic material comprising one of the metal oxides mentioned above into a near-surface layer of the support. Platinum-containing complexes can then be attached to the support surface.

As indicated above, a platinum content in the catalyst may be employed of 1-6 mg platinum/cm³ catalyst, preferably 3-6 or 3-5 mg/cm³. The most preferable platinum content is about 3 mg/cm³. It is believed that a content of about 3 mg/cm³ allows for an even distribution of platinum on the catalyst surface and in its porous interior.

The process described in the present application is conveniently carried out in thermally-controlled reactors (reactor vessels) made of stainless steel. The interiors of the reactors constitute reaction zones which are partitioned by flat-sheet and/or tubular porous supported catalyst elements. Reaction mixtures containing selected quantities of selected alkanes and air and/or oxygen at selected temperatures and pressures are passed through the porous supports in the reaction zones and are then removed from the reactions zones. The condensible reaction products (water, alcohols, aldehydes and carbon dioxide) can be investigated chromatographically.

The benefits of using porous catalytic elements in the flat-sheet or tubular form in the reactors are as follows:
(a) Reduction of reaction rate-limiting factors, such as diffusion of the reaction mixture into catalyst particles, which diffusion plays an important role in conventional fixed-bed granular catalytic systems;
(b) Decrease in the contact time between the reaction mixture and the catalyst; and
(c) Reduction of the possibility of complete alkane oxidation to carbon oxides such as carbon monoxide or carbon dioxide, thus increasing the alcohol and aldehyde selectivity of the process.

Under the conditions described above, the time of contact between the catalyst and the reaction mixture is determined by the velocity of gas flow through the ceramic catalytic elements.

The invention will now be described, by way of non-limiting illustrative example, with reference to the following experiments and with reference to the accompanying Tables, for the catalytic conversion, according to the process of the present invention, of methane to methanol and aldehyde, carried out in the gas phase. The reaction temperature was in the range 30-180° C., preferably 60-120° C., the most preferable temperature range being 90-120° C. The oxidizing agents were air and/or molecular oxygen. The methane:oxygen molar ratio in the reaction mixture was in the range 3-5:1. Mixtures with the ratio 5:1 were found to be the most effective reaction mixtures under the laboratory conditions employed.

Based on the experimental results, it is postulated that the reaction mechanism includes reduction of platinum (IV) to platinum (II) and further to platinum (I). In the presence of air or molecular oxygen the catalyst is spontaneously re-oxidized by molecular oxygen (O₂) to the initial valence state of (IV).

The catalysts which were used are set forth in Table 1, which gives details of catalyst supports, catalyst complexes and platinum content. In Table 2 details of the catalytic activities of the catalysts used in the conversion of methane by oxidation are given. The molar ratio of methane (in natural gas) to oxygen in the reaction mixtures was 3-5:1. The reactions were carried out under atmospheric pressure, and at various temperatures and rates of reaction mixture flow through the reactors. Flat-sheet and tubular catalyst supports were used.

The catalysts described in the present application were found to be active at temperatures lower than those required for activity of homogeneous catalysts containing platinum. The main advantages of the catalysts of the present invention were found to be as follows:
(a) Oxidation by means of the catalytic conversion process was found to be possible in the gas phase;
(b) No additional reagents other than alkane (methane or natural gas) and oxygen or air were found to be necessary for performing the catalytic conversion;
(c) Ease of separation of the heterogeneous catalyst from the reaction products was experienced; and
(d) There was an absence of catalyst contamination of the reaction products.

Changing conditions of the catalytic reaction, the reaction mixture flow rate (residence time) and temperature permitted control of the catalyst selectivity towards the formation of either alcohols or aldehydes (methanol and formaldehyde when methane was oxidized) as shown in Table 3. Optimisation of the catalytic process allowed production of either formaldehyde (selectivity 99%) or methanol (selectivity 86%) as main reaction products at methane conversions of 1-10%.

Table 4 shows that the catalysts of the present invention possess catalytic activity towards light alkanes other than methane present in natural gas. Conversion of ethane and butane (n-butane mixed with iso-butane) significantly exceeded that of methane.

The present invention achieves a degree of conversion and methanol selectivity, in the direct oxidation of methane or natural gas by air or molecular oxygen, which is high. The invention permits direct gas-phase oxidation of lower alkanes to alcohols or aldehydes under mild reaction conditions using molecular oxygen or air. No catalytic promoters of any types, e.g. gaseous chlorine or nitrogen oxides, are required for the process.

TABLE 1
DESCRIPTION OF CATALYSTS

| | | | Content |
|---|---|---|---|
| Cata- | | | of Pt |
| lyst | Support | Complex of Pt | (mg/cm³⁾ |
| 1 | Fibrous oxides: | L = amino, | 1.08 |
| | 47% SiO₂, 40% Al₂O₃, 13% TiO₂ | A = Cl | |
| 2 | Fibrous oxides: | L = imidazolyl, | 1.27 |
| | 47% SiO₂, 46% Al₂O₃, 7% TiO₂ | A = Cl | |
| 3 | Fibrous oxides: | L = hydroxyl, | 1.45 |
| | 50% SiO₂, 42% Al₂O₃, 8% Mo₂O₃ | A = Cl | |
| 4 | Fibrous oxides: | L = amino, | 1.45 |
| | 48% SiO₂, 40% Al₂O₃, 12% Mo₂O₃ | A = amino | |
| 5 | Fibrous oxides: | L = imidazolyl, | 2.50 |
| | 53% SiO₂, 40% Al₂O₃, 7% TiO₂ | A = amino | |
| 6 | Fibrous oxides: | L = hydroxyl, | 3.00 |
| | 53% SiO₂, 40% Al₂O₃, 7% ZrO₂ | A = amino | |
| 7 | Fibrous oxides: | L = amino, | 3.20 |
| | 58% SiO₂, 40% Al₂O₃, 2% ZrO₂ | A = hydroxyl | |
| 8 | Fibrous oxides: | L = imidazolyl, | 5.90 |
| | 57% SiO₂, 40% Al₂O₃, 3% ZrO₂ | A = hydroxyl | |
| 9 | Fibrous oxides: | L = hydroxyl, | 6.00 |
| | 59% SiO₂, 40% Al₂O₃, 1% Mo₂O₃ | A = hydroxyl | |
| 10 | Fibrous oxides: | L = amino, | 6.00 |
| | 47% SiO₂, 53% Al₂O₃ | A = amino | |
| 11 | Fibrous oxides: | L = imidazolyl, | 3.20 |
| | 40% SiO₂, 45% Al₂O₃, 15% ZrO₂ | A = amino | |
| 12 | Tubular porous ceramic: | L = amino, | 2.20 |
| | 70% Al₂O₃, 30% ZrO₂ | A = amino | |

TABLE 2
METHANE OXIDATION ACTIVITY OF DIFFERENT CATALYSTS

| | | T | Flow rate, | Conversion | Selectivity (%) | | |
|---|---|---|---|---|---|---|---|
| Catalyst | Test Gas | (° C.) | (cm³/cm³/min) | of CH₄ (%) | CO₂ | CH₂O | CH₃OH |
| 1 | Natural | 90 | 8.0 | 7.33 | 0.0 | 99.1 | 0.9 |
| | gas | | | | | | |
| 2 | Natural | 90 | 6.7 | 2.54 | 0.8 | 64.2 | 34.7 |
| | gas | | | | | | |
| 3 | Natural | 120 | 6.7 | 3.20 | 2.8 | 94.0 | 3.1 |
| | gas | | | | | | |
| 4 | Natural | 90 | 8.0 | 5.09 | 0.0 | 97.0 | 2.9 |
| | gas | | | | | | |
| 5 | Natural | 90 | 8.0 | 7.78 | 0.0 | 70.1 | 29.2 |
| | gas | | | | | | |
| 6 | Natural | 90 | 8.0 | 4.31 | 0.0 | 97.6 | 3.2 |
| | gas | 120 | 8.0 | 3.26 | 0.0 | 95.2 | 4.5 |
| 7 | Natural | 90 | 8.0 | 2.51 | 0.0 | 12.3 | 87.5 |
| | gas | 120 | 8.0 | 4.68 | 0.0 | 95.0 | 5.3 |
| 8 | Natural | 90 | 8.0 | 0.89 | 0.0 | 36.9 | 63.1 |
| | gas | 120 | 8.0 | 1.35 | 0.0 | 34.6 | 74.8 |
| 9 | Natural | 90 | 8.0 | 1.92 | 0.0 | 34.6 | 65.4 |
| | gas | | | | | | |
| 10 | CH₄ | 90 | 8.0 | 4.70 | 0.0 | 39.5 | 60.5 |
| 11 | CH₄ | 120 | 8.0 | 5.74 | 0.0 | 91.6 | 8.1 |
| 12 | CH₄ | 90 | 8.0 | 15.4 | 4.2 | 65.7 | 30.0 |

TABLE 3
FINAL TESTS OF VARIOUS CATALYSTS IN THE CONVERSION
BY PARTIAL OXIDATION OF METHANE AND NATURAL GAS

| | | T | Flow rate | Conversion of | Selectivity (%) | | |
|---|---|---|---|---|---|---|---|
| Catalyst | Test gas | (° C.) | (cm³/cm³/min) | CH₄ (%) | CO₂ | CH₂O | CH₃OH |
| 2 | Natural | 60 | 22.3 | 0.49 | 0.0 | 91.2 | 8.7 |
| | gas | 90 | | 1.54 | 0.0 | 82.4 | 27.3 |
| | | 120 | | 5.05 | 0.0 | 93.0 | 6.2 |
| | | 60 | 11.2 | 0.12 | 0.0 | 87.2 | 22.4 |
| | | 90 | | 2.12 | 0.0 | 54.8 | 45.2 |
| | | 120 | | 3.89 | 0.0 | 88.4 | 11.5 |
| | | 60 | 6.7 | 1.24 | 0.0 | 80.8 | 18.8 |
| | | 90 | | 2.54 | 0.8 | 64.2 | 34.7 |
| | | 120 | | 1.89 | 2.2 | 82.4 | 15.4 |
| | | 60 | 4.4 | 1.45 | 0.5 | 89.1 | 10.2 |
| | | 90 | | 2.34 | 1.8 | 86.4 | 13.5 |
| | | 120 | | 1.50 | 5.6 | 90.0 | 4.3 |
| | | 60 | 11.2 | 1.35 | 0.0 | 63.0 | 36.7 |
| | | 90 | | 1.53 | 0.0 | 47.5 | 52.4 |
| | | 120 | | 2.31 | 0.0 | 87.5 | 12.3 |
| | | 120 | 6.7 | 3.20 | 2.8 | 94.0 | 3.1 |
| | | 120 | 4.4 | 4.23 | 8.5 | 89.4 | 2.1 |
| 10 | CH₄ | 90 | 22.3 | 3.62 | 0.0 | 99.0 | 0.0 |
| | | 120 | | 2.45 | 0.0 | 99.0 | 0.0 |
| | | 90 | 11.2 | 4.01 | 0.0 | 83.6 | 16.4 |
| | | 120 | | 0.21 | 0.0 | 99.0 | 0.0 |
| | | 60 | 8.0 | 0.54 | 0.0 | 86.5 | 15.4 |
| | | 90 | | 4.70 | 0.0 | 39.5 | 60.5 |
| | | 120 | | 4.62 | 0.0 | 99.0 | 0.0 |
| | | 90 | 6.7 | 0.70 | 0.0 | 17.6 | 82.4 |
| | | 120 | | 5.68 | 0.0 | 89.1 | 10.9 |
| | | 90 | 4.4 | 3.08 | 0.0 | 75.9 | 24.1 |
| | | 120 | | 4.62 | 0.0 | 99.0 | 0.0 |
| 11 | CH₄ | 60 | 80 | 1.76 | 0.0 | 99.2 | 0.0 |
| | | 90 | | 0.98 | 0.0 | 99.5 | 0.0 |
| | | 120 | | 5.74 | 0.0 | 91.6 | 8.1 |
| | | 90 | 6.7 | 3.63 | 0.0 | 92.3 | 17.5 |
| | | 120 | | 1.01 | 0.0 | 13.6 | 86.4 |
| | | 90 | 4.4 | 6.73 | 0.0 | 76.3 | 23.7 |
| | | 120 | | 3.23 | 0.0 | 34.6 | 65.4 |
| | | 90 | 2.2 | 10.22 | 0.0 | 85.9 | 14.0 |
| | | 120 | | 6.11 | 0.0 | 72.8 | 23.2 |

TABLE 4
CONVERSION OF SOME HYDROCARBONS
PRESENT IN NATURAL GAS (% VOL)

| Content in natural gas (% vol) | | |
|---|---|---|
| CH₄ | C₂H₆ | C₄H₁₀ |
| 90.6 | 3.0 | 1.0 |

| | |
|---|---|
| Catalyst | Conversion at 90° C. (%) |

| | | | |
|---|---|---|---|
| 11(1) | 7.3 | 7.3 | 52.5 |
| 22(3) | 2.3 | 3.3 | 61.3 |
| 18(9) | 1.9 | 3.7 | 25.1 |

1. A gas-phase process for the catalytic conversion of a lower alkane comprising at most 5 carbon atoms to at least one product selected from the group consisting of alcohols, aldehydes and mixtures thereof, the process comprising bringing a said lower alkane into contact with an oxidising agent in the presence of a catalyst comprising at least one platinum complex.

2. A process as claimed in claim 1, in which the catalyst is a heterogeneous catalyst, each platinum complex being supported on a catalyst support.

3. A process as claimed in claim 2, in which the catalyst support comprises at least one solid metal oxide, the support having a surface to which each platinum complex is chemically bonded.

4. A process as claimed in claim 3, in which each metal oxide is selected from the group consisting of alumina, silica, titania, zirconia, molybdena and mixtures of any two or more thereof, silicon and molybdenum for this purpose being regarded as metals.

5. A process as claimed in claim 4, in which each catalyst support comprises a plurality of said metal oxides.

6. A process as claimed in claim 3, in which each platinum complex has two ligands which are chemically bonded to the surface of the catalyst support.

7. A process as claimed in claim 6, in which the catalyst has the structure: 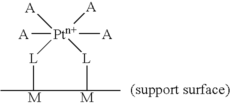
in which:
each L is a ligand selected from amino ligands, hydroxyl ligands and imidazolyl ligands;
each A is a ligand selected from hydroxyl ligands, chloro ligands and amino ligands;
each M is a metal cation of the catalyst support and is selected from the cations of aluminium, silicon, zirconium, titanium and molybdenum; and
n is an integer selected from 4, 2 and 1,
the Pt of the catalyst being a platinum cation having a valence state selected from the valence states (IV), (II) and (1), corresponding to the numerical value of n, said platinum cation forming a central core of the platinum complex, the L ligands and the A ligands surrounding the central core, and the L ligands acting chemically to bond the complex to the surface of the support, under which surface the metal cations M are located.

8. A process as claimed in claim 1, in which the catalyst, including the catalyst support, has a platinum content of 1-6 mg/cm³.

9. A process as claimed in claim 8, in which-the platinum content is 2-5 mg/cm³.

10. A process as claimed in claim 1, in which the lower alkane is methane, the catalytic conversion being direct catalytic conversion by partial oxidation of the methane to form a product selected from the group consisting of methanol, formaldehyde and mixtures thereof.

11. A process as claimed in claim 1, in which the oxidising agent is molecular oxygen, the oxidising agent being contacted in the gas phase with the lower alkane.

12. A process as claimed in claim 11, in which the molecular oxygen is diluted by molecular nitrogen.

13. A process as claimed in claim 1, in which the lower alkane and oxidising agent are mixed together to form a reaction mixture, the reaction mixture being brought into contact with the catalyst at a reaction temperature which is above ambient temperature.

14. A process as claimed in claim 13 in which the reaction temperature is in the range 60-120° C.

15. A process as claimed in claim 13, in which the reaction mixture is brought into contact with the catalyst at ambient atmospheric pressure.

16. A process as claimed in claim 13, in which the catalyst is supported on a solid support contained in a reactor, the reaction mixture being fed into the reactor and into contact with the catalyst at a space velocity of 2-20 cm³ reaction mixture/cm³ reactor volume/minute.

17. A process as claimed in claim 13, in which the catalyst is supported on a solid support contained in a reactor, the support having a porous surface and a porous interior, and the reaction mixture being passed in the reactor over the supported catalyst and through the porous interior of the support.

18. A process as claimed in claim 17, in which the support is in porous sheet or tube form, the support separating a reagent inlet to the reactor from a product outlet from the reactor, the reaction mixture being caused to pass through the porous interior of the support in a direction from the inlet to the outlet.

19. A catalyst for use in a process as claimed in claim 1, the catalyst having the structure: 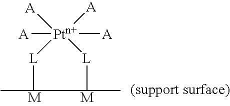
in which:
each L is a ligand selected from amino ligands, hydroxyl ligands and imidazolyl ligands;
each A is a ligand selected from hydroxyl ligands, chloro ligands and amino ligands;
each M is a metal cation of a solid catalyst support comprising at least one metal oxide selected from alumina, silica, zirconia, titania and molybdena, M correspondingly being selected from the cations of aluminium, silicon, zirconium, titanium and molybdenum; and
n is an integer selected from 4, 2 and 1, the Pt of the catalyst being a platinum cation having a valence state selected from the valence states (IV), (II) and (I) and corresponding to the numerical value of n, said platinum cation forming a central core of the platinum complex, the L ligands and the A ligands surrounding the central core, and the L ligands acting chemically to bond the complex to the surface of the support, under which surface the metal cations M are located.

20. A catalyst as claimed in claim 19, in which:
each ligand L is selected from amino ligands and hydroxyl ligands;
each ligand A is selected from hydroxyl ligands and chloro ligands;
each metal cation M is silicon; and
n has a value of 4, the platinum cation of the catalyst having a valence state of (IV).

21. A catalyst as claimed in claim 19, the catalyst support being in porous sheet form, the porosity of the sheet permitting passage of a gas-phase reaction mixture through the sheet from one side of the sheet to another.
